(19) United States
(12) Patent Application Publication
(10) Pub. No.: US 2025/0275708 A1
(43) Pub. Date: Sep. 4, 2025

(54) ELECTROCARDIOGRAM MEASUREMENT SYSTEM COMPRISING PATCH-TYPE ELECTROCARDIOGRAM MEASUREMENT APPARATUS

(71) Applicant: ATsens Co. Ltd., Gyeonggi-do (KR)

(72) Inventors: Deok Byeong CHAE, Gyeonggi-do (KR); Chang Ho LEE, Gyeonggi-do (KR); Jong Ook JEONG, Gyeonggi-do (KR)

(73) Assignee: ATsens Co. Ltd., Gyeonggi-do (KR)

(21) Appl. No.: 19/210,107

(22) Filed: May 16, 2025

Related U.S. Application Data
(63) Continuation-in-part of application No. 18/531,241, filed on Dec. 6, 2023.
Application No. 18/531,241 is a continuation of application No. 16/998,401, filed on Aug. 20, 2020, now Pat. No. 11,872,048.
Application No. 16/998,401 is a continuation of application No. PCT/KR2019/000906, filed on Jan. 22, 2019.

(30) Foreign Application Priority Data
Feb. 22, 2018 (KR) 10-2018-0021064

(51) Int. Cl. A61B 5/339; A61B 5/00; A61B 5/333

(52) U.S. Cl. CPC A61B 5/339; A61B 5/0006; A61B 5/333; A61B 5/6833; A61B 5/746

(57) ABSTRACT

An electrocardiogram measurement system includes a patch-type electrocardiogram measurement apparatus, a first device connected to the electrocardiogram measurement apparatus by means of a first communication and configured to store electrocardiogram data measured by the electrocardiogram measurement apparatus, and a second device connected to the electrocardiogram measurement apparatus by means of the first communication and configured to store electrocardiogram data measured by the electrocardiogram measurement apparatus. The electrocardiogram data measured by the electrocardiogram measurement apparatus includes measured time information.

### CROSS-REFERENCE OF RELATED APPLICATIONS AND PRIORITY

This application is a continuation-in-part application of U.S. patent application Ser. No. 18/531,241, filed Dec. 6, 2023, which is a continuation application of U.S. patent application Ser. No. 16/998,401 (now U.S. Pat. No. 11,872,048), filed on Aug. 20, 2020, which is a continuation of PCT/KR2019/000906, filed on Jan. 22, 2019, which claims the benefit of Korean Patent Application No. 10-2018-0021064 filed on Feb. 22, 2018, the disclosures of which are incorporated herein in their entireties by reference.

### TECHNICAL FIELD

The present disclosure relates to an electrocardiogram measurement system comprising a patch-type electrocardiogram measurement apparatus, and more particularly, to an electrocardiogram measurement system comprising a patch-type electrocardiogram measurement apparatus capable of being connected to an external device to store electrocardiogram data.

### BACKGROUND

Products related to storage of the electrocardiogram (ECG) data known to date are largely divided into products in which data is stored in an embedded memory in a patch-type measurement apparatus itself and products in which data is transmitted to a smartphone through Bluetooth Low Energy BLE communication and stored in a memory in the smartphone, while an ECG signal is output in real time onto the screen of the smartphone.

For a patch-type ECG measurement apparatus using an embedded memory, it is necessary to secure a memory space, which is disadvantageous in that miniaturization of the patch-type ECG measurement apparatus and memory reuse are not allowed. Furthermore, a patch-type ECG measurement apparatus that stores data in the memory of the smartphone has a limitation in that the smartphone must always be carried and connected.

In the prior art, exemplified is an electrode patch for ECG measurement and an ECG measurement device using same disclosed in Korean Application Publication No. 10-2012-0084950 published on Jul. 31, 2012.

### SUMMARY

Provided is an electrocardiogram ECG measurement system comprising a patch-type ECG measurement apparatus in which ECG data is to be stored. Accordingly, it is possible to miniaturize the patch-type ECG measurement apparatus, and is not necessary to always carry a smartphone.

According to an aspect of the present disclosure, an electrocardiogram measurement system includes: a patch-type electrocardiogram measurement apparatus; a first device connected to the electrocardiogram measurement apparatus by means of a first communication method and configured to store the electrocardiogram data measured by the electrocardiogram measurement apparatus; and a second device connected to the electrocardiogram measurement apparatus by means of the first communication method and configured to store the electrocardiogram data measured by the electrocardiogram measurement apparatus.

The subject disclosure describes, among other things, illustrative embodiments of an electrocardiogram measurement system comprising: an electrocardiogram measurement apparatus configured to measure electrocardiogram data by means of one or more electrodes, amplify the electrocardiogram data, and convert digitally the electrocardiogram data; a first device comprising a first processor, a second communication unit, a third communication unit and a first memory; and a second device comprising a second processor, a fourth communication unit, a fifth communication unit and a second memory, the second device communicating with the first device through a wireless communication in response to a request of the first device. The electrocardiogram measurement apparatus includes a first communication unit and is further configured to transmit, through the first communication unit, the amplified and digitized electrocardiogram data to the first device, or alternatively transmit the amplified and digitized electrocardiogram data to the second device. The first device is configured to: receive first electrocardiogram data and measured time information associated with the first electrocardiogram data from the electrocardiogram measurement apparatus via the second communication unit, store the received first electrocardiogram data in the first memory for a first time period, display, on a user interface of the first device, the received first electrocardiogram data; upon selection of the second device, transmit, to the second device, a command for making a connection with the electrocardiogram measurement apparatus via the third communication unit, and release a connection from the electrocardiogram measurement apparatus after the transmitting the command for making the connection to the second device. The second device is configured to: receive second electrocardiogram data and measured time information associated with the second electrocardiogram data from the electrocardiogram measurement apparatus via the fourth communication unit, in response to the command for making the connection with the electrocardiogram measurement apparatus from the first device via the fifth communication unit, and store, in the second memory, the received second electrocardiogram data for a second time period. The electrocardiogram measurement apparatus is further configured to transmit the second electrocardiogram data to the second device via the first communication unit while the electrocardiogram measurement apparatus is not communicating with the first device; the first time period is a time period during which the first device is connected with the electrocardiogram measurement apparatus; and the second time period is a time period during which the second device communicates with the electrocardiogram measurement apparatus and the first device is disconnected from the electrocardiogram measurement apparatus.

The subject disclosure describes, among other things, illustrative embodiments of a method of operating an electrocardiogram measuring system comprising an electrocardiogram measurement apparatus, a first device capable of communicating with the electrocardiogram measurement apparatus, and a second device capable of communicating with the electrocardiogram measurement apparatus. The method includes receiving, by a first device comprising a first processor and a first memory, first electrocardiogram data and measured time information associated with the first electrocardiogram data from an electrocardiogram measurement apparatus for a first time period, and storing the received first electrocardiogram data in a first memory of the first device; displaying, by the first device, the first electrocardiogram data on a screen thereof; transmitting, to a second device, by the first device, a set of commands for making a connection with or releasing the connection from the electrocardiogram measurement apparatus through a wireless communication method, wherein the second device is selected to store the electrocardiogram data; connecting, by the second device including a second processor and a second memory, to the electrocardiogram measurement apparatus in response to the set of commands for making the connection with the electrocardiogram measurement apparatus through the wireless communication method; receiving, by the second device, a second electrocardiogram data and measured time information associated with the second electrocardiogram from the electrocardiogram measurement apparatus for a second time period, and storing the second electrocardiogram data in a second memory of the second device. The electrocardiogram measurement apparatus is configured to measure the first electrocardiogram data and the second electrocardiogram data, by means of one or more electrodes, amplify the electrocardiogram data, convert digitally the first and the second electrocardiogram data, and transmit the amplified and digitized first electrocardiogram data to the first device, or alternatively transmit the amplified and digitized second electrocardiogram data to the second device, through a first communication unit of the electrocardiogram measurement apparatus. The method further includes receiving, by the second device, the second electrocardiogram data from the electrocardiogram measurement apparatus, while the electrocardiogram measurement apparatus is not communicating with the first device. The first time period is a time period during which the first device is connected with the electrocardiogram measurement apparatus, and the second time period is a time period during which the second device is connected with the electrocardiogram measurement apparatus which does not communicate with the first device. The second device communicates with the first device through the wireless communication method in response to a request of the first device.

According to an aspect of the present disclosure, an electrocardiogram measurement system includes: a patch-type electrocardiogram measurement apparatus; a first device connected to the electrocardiogram measurement apparatus by means of a first communication method and configured to store the electrocardiogram data measured by the electrocardiogram measurement apparatus; and a second device connected to the electrocardiogram measurement apparatus by means of the first communication method and configured to store the electrocardiogram data measured by the electrocardiogram measurement apparatus.

Furthermore, the electrocardiogram data measured by the electrocardiogram measurement apparatus may include measured time information. A first time period in which the first device stores the electrocardiogram data measured by the electrocardiogram measurement apparatus, and a second time period in which the second device stores the electrocardiogram data measured by the electrocardiogram measurement apparatus may be at least partially at different times.

Specifically, the first device may receive and store the electrocardiogram data stored in the second device that has been measured by the electrocardiogram measurement apparatus. The first device may combine the electrocardiogram data stored in the first device and the electrocardiogram data received from the second device using the measured time information included in each piece of the electrocardiogram data.

In addition, when the second device is connected and the first device displays the electrocardiogram data through an application program installed in the first device, the first device may combine and display the electrocardiogram data stored in the first device and the electrocardiogram data received from the second device using the measured time information included in each piece of the electrocardiogram data.

Furthermore, in a state where the second device is connected with the electrocardiogram measurement apparatus by means of the first communication method, the first device may be characterized by instructing the second device to release the connection from the electrocardiogram measurement apparatus when the first device is in a state of being able to be connected with the electrocardiogram apparatus.

In a state where the second device is able to be connected with the electrocardiogram measurement apparatus by means of the first communication method, the first device may be characterized by instructing the second device to be connected with the electrocardiogram measurement apparatus.

In addition, when there is no external device that is able to receive the electrocardiogram data measured in the electrocardiogram measurement apparatus, the electrocardiogram measurement apparatus may generate a warning signal. Furthermore, when the first device is unable to receive the electrocardiogram data measured in the electrocardiogram measurement apparatus, and the second device is also unable to receive the electrocardiogram data measured in the electrocardiogram measurement apparatus, the first device may generate a warning signal.

According to the electrocardiogram ECG measurement system comprising the patch-type ECG measurement apparatus of the present disclosure, a device in which ECG data is to be stored may be selectively used, and thus, it is possible to miniaturize the patch-type ECG measurement apparatus and is not necessary to always carry the smartphone and keep in connection.

### BRIEF DESCRIPTION OF DRAWINGS

FIG. 1 is a configuration diagram of an electrocardiogram ECG measurement system comprising a patch-type ECG measurement apparatus according to an embodiment of the present disclosure;

FIG. 2 is a configuration diagram of an ECG measurement apparatus according to an embodiment of the present disclosure;

FIG. 3 is a configuration diagram of a first device according to an embodiment of the present disclosure;

FIG. 4 is a configuration diagram of a second device according to an embodiment of the present disclosure; and

FIG. 5 is an illustration of a combination of ECG data in the first device.

FIG. 6 is a flowchart of a method for processing placement value data of ECG data before the start of measurement according to one embodiment of the present disclosure.

FIG. 7 is a flowchart of a method for processing placement value data of ECG data during measurement according to one embodiment of the present disclosure.

FIG. 8 is a non-limiting example of a user interface in which ECG data and placement value values are displayed according to one embodiment of the present disclosure.

FIG. 9 is a schematic illustration of a process of measuring ECG data while changing the placement position of the ECG measurement apparatus.

FIG. 10 is a schematic illustration of a process of measuring ECG data by the ECG measurement apparatus at each placement position.

FIG. 11 illustrates a method for receiving and processing ECG data measured at multiple locations.

FIG. 12 illustrates a non-limiting example of a user interface that displays ECG data and placement value values for the ECG data while being attached to multiple locations.

FIG. 13A is a schematic illustration of a process in which a placement state is calculated and displayed according to an embodiment of the present disclosure.

FIG. 13B is a schematic illustration of a process of showing features detected by the placement determination module.

### DETAILED DESCRIPTION

Hereinafter, an electrocardiogram ECG measurement system comprising a patch-type ECG measurement apparatus according to an embodiment of the present disclosure will be described in detail with reference to the accompanying drawings.

The patch-type electrocardiograph device according to one embodiment of the present disclosure may be attached to a part of the body of a subject, for example, the heart, to measure electrocardiogram signals for 48 hours or 14 days. Electrocardiogram signals measured over a longer period of time may increase the accuracy of diagnosis and/or prediction of diseases such as arrhythmia. Here, the subject may be a person or an animal.

A patch-type electrocardiograph device may be implemented with a flexible material that may be deformed to conform to the curved surface of the subject's body, for example, by including an elastic, i.e. stretchable, cloth. A patch-type electrocardiograph device may detect electrical potentials generated on the body surface by contacting the body surface. The electrocardiogram signal measured by a patch-type electrocardiograph is measured on the surface of the body by detecting electrical signals from the heart's movements, and the measurement values may differ depending on the location and direction of the patch. Even for electrocardiogram signals measured from the same subject, the measurement values, patterns, and shapes may differ each time they are measured. Since the position of the heart differs for each subject and the distribution of internal organs such as muscles, blood vessels, and bones also differs, the position and direction of placement of the electrocardiograph device, which measures the electrocardiogram signal, may also differ.

In the present embodiment, the electrocardiogram signal includes an electrocardiogram measured over time, and may be a discrete signal or a continuous signal.

In the embodiment of this disclosure, the measured ECG signal may vary depending on the location or orientation of the placement. ECG signals measured from the same subject may also vary unless they are attached at the same location and/or orientation.

The following embodiments of the present disclosure are intended to embody the present disclosure, but not to limit or restrict the scope of the present disclosure. From the detailed description and embodiments of the present disclosure, all techniques easily conceivable by those skilled in the art to which the present disclosure pertains may be easily interpreted as belonging to the scope of the present disclosure.

The term “peak value” as used herein refers to a value for a signal peak existing in an electrocardiogram signal. The “peak value” may be substituted with “value of peak”. The term “peak value” as used herein refers to the time-domain positions or corresponding amplitudes of peaks identified in an electrocardiogram (ECG) signal, wherein the peaks correspond to characteristic waveform components including the P wave, Q wave, R wave, S wave, and T wave. Each peak value may include a timestamp indicating the temporal location of the peak within the ECG signal, or an amplitude value representing the signal strength at the corresponding time. The detected peak values may be obtained by applying one or more signal processing algorithms to the ECG signal, such as bandpass filtering, derivative filtering, squaring, and moving window integration, followed by peak detection techniques such as the Pan-Tompkins algorithm or wavelet-based methods.

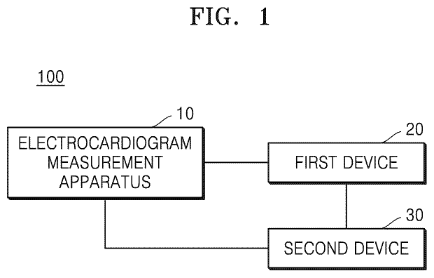

First, FIG. 1 is a configuration diagram of an ECG measurement system 100 comprising a patch-type ECG measurement apparatus according to an embodiment of the present disclosure.

As may be known from FIG. 1, the ECG measurement system 100 according to the embodiment of the present disclosure includes an ECG measurement apparatus 10, a first device 20, and a second device 30.

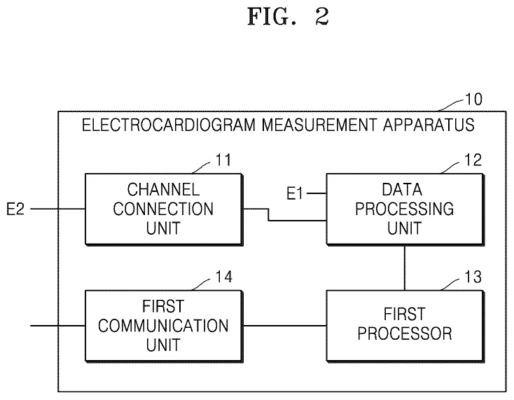

FIG. 2 is a configuration diagram of the ECG measurement apparatus 10 according to an embodiment of the present disclosure.

As may be known from FIG. 2, the ECG measurement apparatus 10 may be configured by including a plurality of electrodes El, a channel connection unit 11, a data processing unit 12, a first processor 13, and a first communication unit 14. The ECG measurement apparatus 10 may further include a heart rate sensor, a respiration sensor, a temperature sensor, and a heart sound sensor, as needed.

The ECG measurement apparatus 10 has the shape of a patch type, and is attached to a human body to be able to measure ECG data of one or more channels by means of the plurality of electrodes E1. A plurality of electrodes E1 may be applied to measure the electrical potential associated with heartbeats on the body surface or to inject an electrolyte drug into the body through the user's skin or mucous membranes. The plurality of electrodes E1 may preferably be provided with two or more electrodes. In addition, the number of ECG measurement channels of the ECG measurement apparatus 10 is possibly extended by receiving the ECG measurement data of the one or more channels, which is measured by means of the external electrodes E2, through the channel connection unit 11. As the channel connection unit 11, an input terminal form may be exemplified which enables the measurement data from the external electrodes E2 to be used as an input of the data processing unit 12.

The ECG data measured by means of the electrodes E1 of the ECG measurement apparatus 10 itself or the external electrodes E2 is amplified by the data processing unit 12 and then converted into a digital signal. To this end, the data processing unit 12 preferably includes an amplifier and an analog-to-digital converter. The ECG data may have different signal patterns and measured values depending on the location where it is attached.

Furthermore, the ECG data output from the data processing unit 12 is inserted with time information at which the ECG data has been measured by the first processor 12, namely, a time-stamp, and then transmitted to an external device by means of a first communication method through the first communication unit 14. Specifically, the first communication method may be exemplified by a Bluetooth low energy BLE communication. The ECG measurement apparatus 10 may transmit the ECG data to the external device and receive data from the external device through the first communication unit 14. Here, for convenience, the description is provided only with the ECG data, but it is natural to include a necessary control signal together with the data. The ECG measurement apparatus 10 may transmit the bio-signals measured by the heart rate sensor, respiratory sensor, temperature sensor, and heart sound sensor as external data.

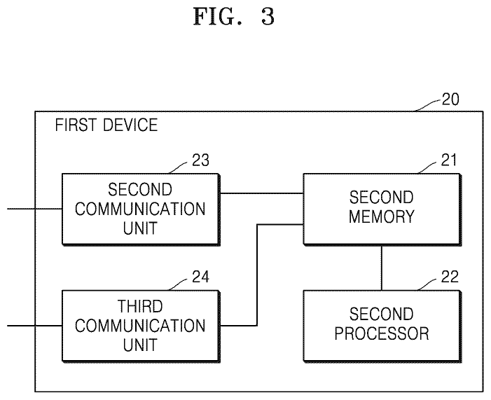

FIG. 3 is a configuration diagram of the first device 20 according to an embodiment of the present disclosure.

As may be known from FIG. 3, the first device 20 may be configured by including a second memory 21, a second processor 22, a second communication unit 23, and a third communication unit 24. For example, the first device 20 may be implemented using a mobile terminal such as a smartphone, tablet PC, PDA Personal Digital Assistants, PMP Portable Multimedia Player, digital broadcasting terminal, or laptop computer, but may not be limited to these and may be any device including a processor, memory, and communication unit.

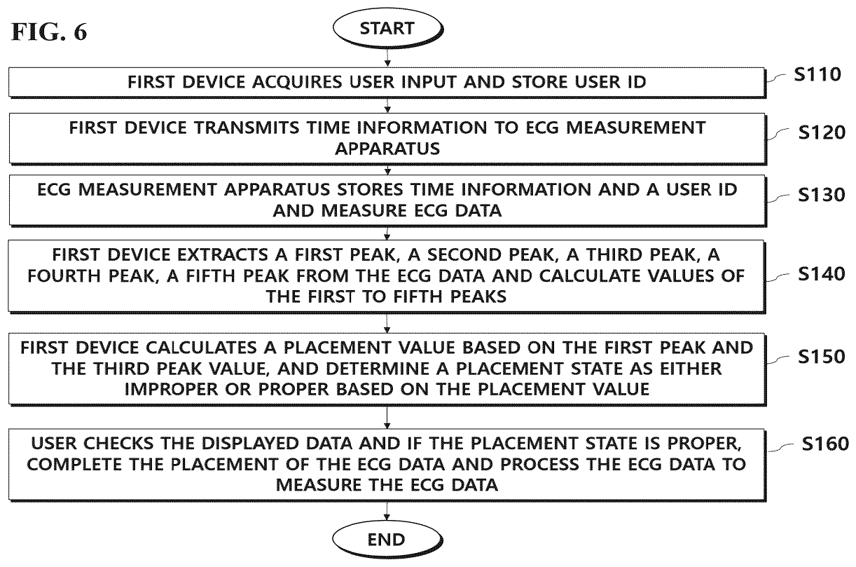

The second memory 21 may store the ECG data measured by the ECG measurement apparatus 10. The first device 20 is installed with an application program, and thus the second processor 22 may execute the application program. When the application program is executed by the second processor 22, the ECG data stored in the second memory 21 may be processed to be displayed on a screen of the first device 20. The first device 20 may ensure that the ECG measurement apparatus 10 is correctly positioned. When the ECG measurement apparatus 10 is placed in the proper position, the ECG measurement apparatus 10 may record an ECG signal that includes a normal signal pattern. Having a normal signal pattern may mean that the R-wave is periodically detected in the ECG signal and that the PQRST waves are present in the ECG signal. The first device 20 may determine, according to the method of FIG. 6, whether the ECG data has a normal signal pattern, whether the placement of the ECG measurement apparatus 10 is proper. The first device 20 may identify the ECG data received in real time to determine whether the ECG measurement apparatus 10 is placed in a correct position and display information on a determined placement value. Here, the determined placement value refers to an evaluation value of the position where the ECG patch device is placed. The determined placement value may be a numerically derived value that indicates how proper the given position is for measuring the ECG signals generated by the user's heart. The determined placement value may be calculated using a predefined mathematical formula, with the peak values from the ECG signal as inputs. The first device 20 may identify the ECG data received in real time to determine whether the ECG measurement apparatus 10 is placed properly, and transmit a placement state including the placement value to an external device or the ECG measuring apparatus 10. The first device 20 may transmit a request of an alert about the placement state to output the alert in the ECG measurement apparatus 10. The first device 20 may generate data related to the alert at a timing associated with an occurrence of the alert. The alert may be triggered upon a change in the placement state, such as a transition from a proper state to an improper state. The timing for generating the alert may be determined based on user-input data. The ECG measurement apparatus 10 may output an alert visually or audibly based on the placement value or the placement state. In another embodiment, the first device 20 may store the placement data for the ECG data as separate placement value with a time stamp. For example, for ECG data, if the placement value from the first time to the second time is determined to be improper, data including the first time, the second time, and improper may be stored. In this way, the placement value for the time period in which the placement value is improper may be stored. The first device 20 may store placement value together with ECG data. The first device 20 may process the placement value to be displayed together with the ECG data. For example, the first device 20 may display the ECG data while displaying whether a placement state of the placement value was proper or improper in a separate position. If the ECG measurement apparatus 10 detects that its placement state is proper based on the ECG data measured, the first device 20 or the second device 30 may display a message to start ECG measurement.

The second communication unit 23 receives the ECG data from the ECG measurement apparatus 10 by means of the first communication method. The first communication method may be exemplified by the BLE communication. The ECG data received by the second communication unit 23 is stored in the second memory 21. The ECG data received from the ECG measurement apparatus 10 is characterized by being inserted with the time information at which the ECG data has been measured, namely, the time-stamp.

The third communication unit 24 enables transmission and reception of data with the second device by means of a wireless or wired communication method other than the first communication method. The third communication unit 24 may use a WiFi communication as another example. Naturally, the second device 30 and the first device 20 may be directly connected through a USB terminal of the first device 20 and then data is input from or output to the second device 30. The ECG data input from the second device 30 through the third communication unit 24 or a direct connection is stored in the second memory 21.

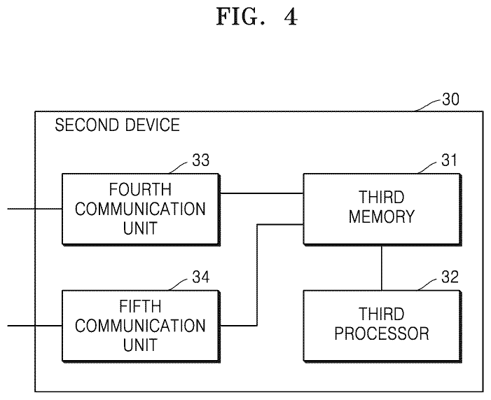

FIG. 4 is a configuration diagram of the second device 30 according to an embodiment of the present disclosure.

As may be known from FIG. 4, the second device 30 may be configured by including a third memory 31, a third processor 32, a fourth communication unit 33, and a fifth communication unit 34. As an example, the second device 30 is preferably implemented with a device miniaturized by mounting a communication function onto a device with a memory function, such like a dongle memory. The second device 30 may be implemented with a device by mounting other device.

The third memory 31 may store the ECG data measured by the ECG measurement apparatus 10. The third processor 32 may execute processing or the like for the ECG data.

The fourth communication unit 33 receives the ECG data from the ECG measurement apparatus 10 by means of the first communication method. The first communication method may be exemplified by the BLE communication. The ECG data received by the fourth communication unit 33 is stored in the third memory 31. The ECG data received from the ECG measurement apparatus 10 is characterized by being inserted with the time information at which the ECG data has been measured, namely, the time-stamp.

The fifth communication unit 34 enables transmission and reception of data with the first device 20 by means of a wireless communication method other than the first communication method. The fifth communication unit 34 may use a WiFi communication as an example. Naturally, the second device 30 and the first device 20 may be directly connected through a USB terminal of the first device 20 and thus data transmission and reception with the second device 30 become possible. While communicating with the ECG measurement apparatus 10 to receive ECG data, the second device 30 may indicate whether the ECG data has a normal pattern. The second device 30 may determine, according to the method of FIG. 6, whether the ECG data has a normal pattern (including PQRST waves) and/or whether the placement of the ECG measurement apparatus 10 is proper. The first device 20 may identify the ECG data received in real time to determine whether the ECG measurement apparatus 10 is attached properly and display information on the determined proper condition (placement value or placement state). The second device 30 may identify the ECG data received in real time to determine whether the ECG measurement apparatus 10 is attached properly, and transmit the information on the determined proper state to an external device or the ECG measurement apparatus 10. The ECG measurement apparatus 10 may output a predetermined alert visually or audibly based on the received information on the fitness status. Here, the alert can output one of two states: proper or improper, for the placement value of the ECG measurement apparatus 10. In the present specification, the term “proper” may refer to a state in which the measured ECG signal is sufficient to determine the condition of the heart. Conversely, the term “improper” may refer to a state in which the measured ECG signal is insufficient to determine the condition of the heart. The criterion value for determining whether the state is a “proper state” or an “improper state” may vary depending on the ECG signal analysis technique or algorithm.

In another embodiment, the second device 30 may store the fitness status information of the placement value for the ECG data as separate placement value data with a time stamp. For example, for ECG data, if the placement value from the first hour to the second hour is determined to be inappropriate, data for {first hour, second hour, inappropriate} may be stored. In this way, data for the time period in which the placement value is inappropriate may be stored. The second device 30 may store the placement value data together with the ECG data. The second device 30 may process the placement value data to be displayed together with the ECG data. For example, the second device 30 may display the ECG data while displaying in a separate position whether the placement value was appropriate or inappropriate.

A proper placement value may indicate that the user's heart state is suitable for diagnosis based on the measured ECG signal. An improper placement value may indicate that the user's heart state is unsuitable for diagnosis based on the measured ECG signal. An improper placement may mean that the ECG measurement apparatus is incorrectly positioned. Ideally, if the P wave in the ECG signal is clearly visible in the measured ECG signal, the placement value calculated may be considered to be proper. An improper placement may mean that the P wave is not measured, or that the magnitude between the PR waves is not accurate.

When communication with the ECG measurement apparatus 10 is terminated, the second device 30 may transmit the ECG data and the placement value regarding to the ECG data to the first device 20. The transmitted data may include the ECG data measured, and each placement value measured at a predetermined time period. Each placement may be recorded as data along with a timestamp. The timestamp may represent a start time or a time within the time period.

Hereinafter, a detailed description will be provided about a method for storing the ECG data, which has been measured by the ECG measurement apparatus 10, in the first device 20 and the second device 30.

The first device 20 preferably operates as a master device that may transmit a command to the second device 30 to control the operation of the second device 30. In other words, the second device 30 is characterized by operating as a slave device. Accordingly, the first device 20 may not only receive connection status information about the second device 30 with another device, but also transmit a command for connecting the second device 30 with the other device or a command for releasing the connection of the second device 30 from the other device. Here, the other device may be the ECG measurement apparatus 10.

When the first device 20 is connected to the ECG measurement apparatus 10 by means of the first communication method, the ECG data measured by the ECG measurement apparatus 10 is stored in the first device 20.

In addition, when the connection of the first device 20 with the ECG measurement apparatus 10 through the first communication method is released, the second device 30 may be automatically connected to the ECG measurement apparatus 10 when the second device 30 is present around the ECG measurement apparatus 10. Accordingly, the ECG data measured by the ECG measurement apparatus 10 is stored in the second device 30.

In other words, a time period in which the first device 20 stores the ECG data measured by the ECG measurement apparatus 10, and a time period in which the second device 30 stores the ECG data measured by the ECG measurement apparatus 10 are different from each other, and thus the time periods do not overlap. In other words, the time period in which the first device 20 stores the ECG data measured by the ECG measurement apparatus 10, and the time period in which the second device 30 stores the ECG data measured by the ECG measurement apparatus 10 are characterized by being at different times. This is caused by the characteristics of the BLE communication in which only one device may be connected to one communication module. Furthermore, even when a communication in which many devices are connected at the same time is used, namely, even when the first device 20 and the second device 30 simultaneously store the ECG data, overlapping ECG data may be discerned by means of the time information, namely, the time-stamp. To sum up, the time period in which the first device 20 stores the ECG data measured by the ECG measurement apparatus 10, and the time period in which the second device 30 stores the ECG data measured by the ECG measurement apparatus 10 may be at least partially at different times.

The first device 20 may receive and store the ECG data that has been measured by the ECG measurement apparatus 10 and stored in the second device 30. The first device 20 and the second device 30 may be exemplarily connected by means of a wireless communication method other than the first communication method, or a direct connection.

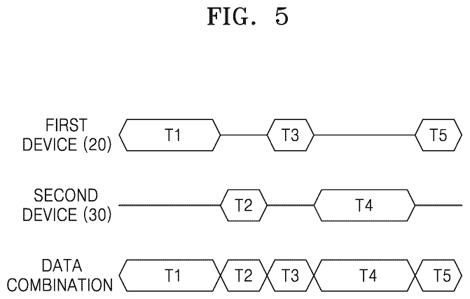

FIG. 5 is an illustration of a combination of the ECG data in the first device 20. Here, for convenience of explanation, the ECG data is shown not overlapping, but it is natural that the ECG data stored in the first device 20 and the ECG data stored in the second device 20 at least partially overlap. This overlapping portion may be discerned by means of the time-stamp that is the characteristics of the present disclosure.

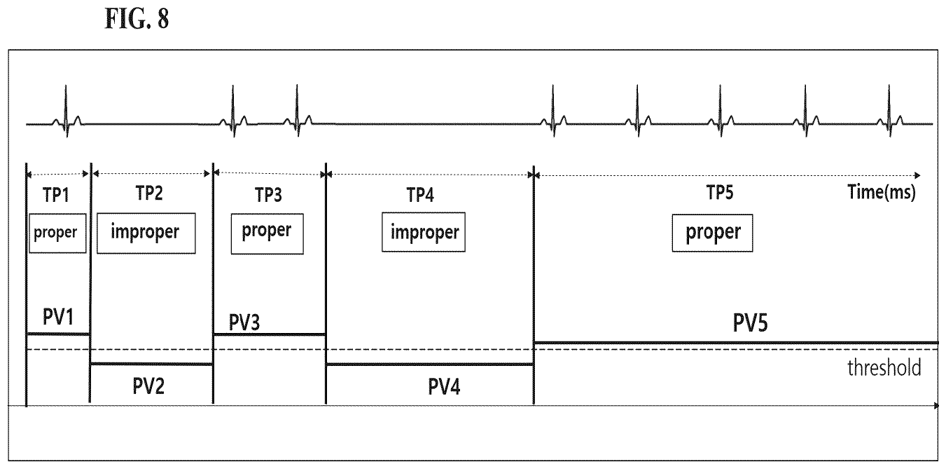

The first device 20 combines the ECG data, which has been measured by the ECG measurement apparatus 10 and stored in the first device 20, and the ECG data, which has been measured by the ECG measurement apparatus 10 and stored in the second device 30, using the measured time information included in each piece of the ECG data, and then newly store the combined ECG data in the first device 20. The first device 20 may store placement data by combining placement values measured across multiple time intervals with the ECG data using the measured timestamp, as shown in FIG. 8.

In detail, when the second device 30 is connected to the ECG measurement apparatus 10 and the ECG data is processed displayed as an example through an application program installed in the first device 20, the first device 20 is characterized by combining the ECG data, which has been measured by the ECG measurement apparatus 10 and stored in the first device 20, and the ECG data, which has been measured by the ECG measurement apparatus 10 and stored in the second device 30, using the measured time information included in each piece of the ECG data, and displaying the combined ECG data on the screen of the first device 20. As shown in FIG. 8, the screen of the first device 20 may display the combined ECG data and placement values (PV1, PV2, PV3, etc.) using the measured time intervals.

The first device 20 may select one of the first device 20 or the second device 30 and allow the ECG data measured by the ECG measurement apparatus 10 to be stored therein. Hereinafter, a method in which the first device 20 selects one of the first device 20 or the second device 30 will be described.

In a state where the second device 30 is connected with the ECG measurement apparatus 10 by means of the first communication method, when the first device 20 is in a state of being able to be connected to the ECG measurement apparatus 10 by means of the first communication method, the first device 20 instructs the second device 30 to release the connection from the ECG measurement apparatus 10. Accordingly, the connection between the second device 30 and the ECG measurement apparatus 10 is released, the first device 20 is connected with the ECG measurement apparatus 10, and thus the ECG data measured in the ECG measurement apparatus 10 is stored in the first device 20.

Here, one example is that the first device 20 is connected with the ECG measurement apparatus 10 in a wired manner and the second device 30 is connected with the ECG measurement apparatus 10 in a wirelessly manner. When the second device 30 lose the ECG data due to wireless disconnection, the lost data may be recovered by communicating with the first device 20 with time-stamp information.

In addition, when the second device 30 is in a state of being able to be connected to the ECG measurement apparatus 10 by means of the first communication method, the first device 20 may instruct the second device 30 to be connected with the ECG measurement apparatus 10. As an example, in a state where the first device 20 is connected with the ECG measurement apparatus 10 by means of the first communication method, when the first device 20 intends to stop the storage of the ECG data, the first device 20 may release the connection of itself from the ECG measurement apparatus 10, and allow the second device 20 to be connected with the ECG measurement apparatus 10. However, when the first device 20 releases the connection of itself with the ECG measurement apparatus 10, the second device 30 may be automatically connected with the ECG measurement apparatus 10 when the second device 30 is present around the ECG measurement apparatus 10. The first device 20 or the second device 30 connected to the ECG measurement apparatus 10 may receive ECG data and determine whether the placement value is appropriate or proper in real time, thereby generating placement data including a placement value and a placement state. If a placement state of a placement value is not proper, the first device 20 or the second device 30 may send a message to the ECG measurement apparatus 10 or an external device that the placement state is not proper. The first device 20 or the second device 30 may periodically transmit a message including a placement data including a placement value. The message may include an event indicating that the placement state has changed from being proper to being improper or an event indicating that the placement state has changed from being improper to being proper. Here, the external device may be a user terminal carried by the tester or a user terminal carried by the medical staff, but the external device may also be a computing device.

The first device 20 or the second device 30 may output the time values and measured values of the peaks in the ECG data using a peak detection module. The peak detection module may find the P, Q, R, S, and T peaks in the ECG data and convert the P, Q, R, S, and T peaks into data. The peak detection module may be configured to detect the P, Q, R, S, and T peaks in the ECG signal based on the features of each respective peak. The first device 20 or the second device 30 may output data including a placement value or a placement state from the ECG data using a placement determination module. The placement determination module may be configured to output the placement value corresponding to the ECG data by inputting at least one of the values of the P, Q, R, S, and T peaks.

The placement value is a value determined by the ECG signal measured by the ECG measurement apparatus 10 while placed to the human body, and may be a value corresponding to how accurately the electrical signal corresponding to the movement of the heart is measured. If the placement value is below a predetermined threshold, the placement state may be set to improper. If the placement value is equal to or above the threshold, the placement state may be set to proper. The placement state is proper when the ECG signals corresponding to the heart movement are measured at a level higher than the predetermined threshold. During TP1, TP3, and TP5 (See FIG. 8), the P, Q, R, S, and T peaks are measured accurately, so the trend of the user's heart movement may be accurately determined. In other words, the ECG data measured during TP1, TP3, and TP5 plays an important role in determining, predicting, and inferring the user's heart state. The user's heart state may include the possibility of arrhythmia, atrial fibrillation, and other related states.

An ECG data set that has been measured in an improper position will not be accurately matched with the user's heartbeat, even if it is analyzed. Even if the ECG measurement apparatus 10 is placed to a same position, the placement state of the ECG signal measured from a first user may be set to proper, while the placement state of the ECG signal measured from a second user may be set to improper. Due to differences in heart position, muscle, and fat, the position where the placement state is proper for the first user may differ from the position where the placement state is proper for the second user. The improper position is a state in which at least one of the P, Q, R, S, and T peaks, which correspond to the heart's movements, is not measured accurately, or in which it is difficult to measure the P, Q, R, S, and T peaks accurately. The ECG data measured in an improper position may not be used to determine, predict, or infer the user's heart disease. The ECG data measured in an improper position may lead to incorrect determination, prediction, or inference of the user's heart disease.

The first device 20 or the second device 30 may receive ECG data by communicating with the ECG measurement apparatus 10 applied to the patient in the hospital and output the placement value or the placement state. The first device 20 or the second device 30 may receive ECG data by communicating with a remote ECG measurement apparatus 10 that communicates via networking and output the placement value.

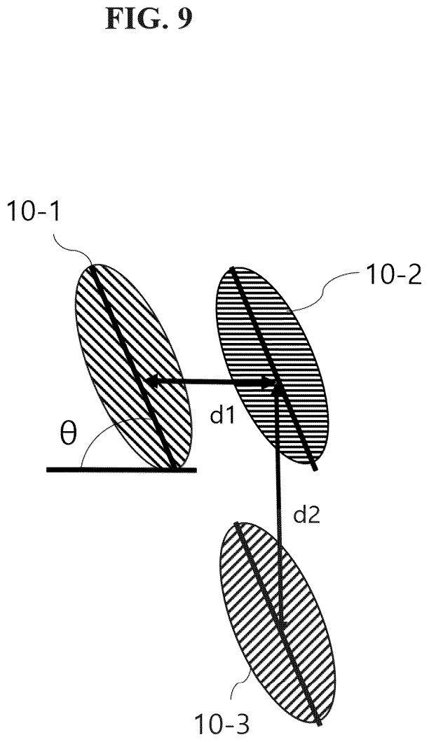

More specifically, when the user applies an ECG measurement apparatus 10, the ECG measurement apparatus 10 may send a signal to the first device 20 and the second device 30 to notify them to start measuring, and attach the ECG measurement apparatus 10 to different positions such as a first position, a second position, and a third position at a set time interval so that electrocardiogram signals may be measured. As shown in FIG. 9, the user changes the placement position of the ECG measurement apparatus 10 to a first position 10-1, a second position 10-2, and a third position 10-3 so that ECG data is measured at the positions.

In the first time period, ECG data is measured by being attached to the first position, in the second time period, ECG data is measured by being attached to the second position, and in the third time period, ECG data is measured by being attached to the third position, and the first device 20 or the second device 30 may receive the ECG data. The first device 20 or the second device 30 outputs the first placement value for the ECG data attached to the first position in the first time interval, the second placement value for the ECG data attached to the second position in the second time interval, and the third placement value for the ECG data attached to the third position in the third time interval, and the first placement value, The best value among the second placement value and the third placement value may be selected. The first device 20 or the second device 30 may display the selected information. Information on the best value among the first placement value, the second placement value, and the third placement value may be sent to the user's device. The user may measure the ECG data by attaching the ECG measurement apparatus 10 to the position with the best value among the first to the third positions.

When there is not any external device including the first device 20 or the second device 30 that may receive the ECG data measured by the ECG measurement apparatus 10, the ECG measurement apparatus 10 generates a warning signal to inform a user of a situation in which the ECG data is not possibly stored. The ECG measurement apparatus 10 may receive a message indicating an improper state from the first device 20 or the second device 30 and generate and output an alert corresponding to the improper state. If the improper state for a certain period of time, the ECG measurement apparatus 10 may stop measuring the ECG signal in the improper state. The ECG measurement apparatus 10 may halt ECG signal measurement until the placement state changes to proper. Furthermore, when the first device 20 is not able to receive the ECG data measured by the ECG measurement apparatus 10, and the second device 20 is not also able to receive the ECG data measured by the ECG measurement apparatus 10, the first device 20 may generate a warning signal. In other words, the warning signal in the present disclosure may be generated by the ECG measurement apparatus 10 and/or the first device 20.

Referring back to FIG. 1, according to the ECG measurement system 100 comprising a patch-type ECG measurement apparatus 10 of the present disclosure, any one of the first device 20 and the second device 30 may be selectively used as a device in which the ECG data is to be stored. Accordingly the patch-type ECG measurement apparatus 10 does not require a separate and embedded large-capacity memory, and thus may be miniaturized.

In addition, according to the ECG measurement system 100 comprising the patch-type ECG measurement apparatus 10 of the present disclosure, it is not necessary to always carry the first device 20 of a large size like a smartphone, and the ECG data may be stored in the second device 30 of a small dongle type. Furthermore, according to the ECG measurement system 100 comprising the patch-type ECG measurement apparatus 10 of the present disclosure, pieces of the ECG data stored in different devices at different times are combined to one piece of data using the measured time information and thus the entire ECG data may be constructed, which increases the convenience of use.

FIG. 6 is a flowchart of a method for processing a placement value of ECG data before the start of measurement according to one embodiment of the present disclosure.

Referring back to FIG. 1, in the electrocardiogram measurement system 100, the placement value may be determined whether a placement state of the ECG measurement apparatus 10 is appropriate or not based on the ECG data measured by the ECG measurement device 10 before the electrocardiogram measurement. The ECG data measured on the surface of the body according to the signals from the heart's movements may be deformed depending on a location where it is attached. Since a physical state, position, shape, size, etc. of organs such as muscles, blood vessels, and bones are different, as well as the position of the heart, the ECG data may be deformed differently depending on the location where it is attached.

For the sake of convenience, the first device 20 is described in the following description, but instead of the first device 20, the second device 30 can be used.

In S110, the first device 20 may acquire user input and store a user ID. The first device 20 may acquire and store the user ID through a user interface that acquires a user ID. The first device 20 may transmit the user ID to the ECG measurement apparatus 10.

In S120, the first device 20 may transmit time information to the ECG measurement apparatus 10. The first device 20 may generate the time information by a timer that internally generates a clock signal. Here, the timer may be a hardware timer and may be designed to continuously generate signals by existing inside the processor. In another embodiment, the first device 20 may generate the time information by synchronizing with an external time server. The first device 20 may be synchronized with an external time server via the Internet. The first device 20 may share a time value of the time information by sending a current time value of the timer to the ECG measurement apparatus 10 so that the time value of the ECG measurement apparatus 10 is set to the current time value.

In S130, the ECG measurement apparatus 10 may store the time value and the user ID and measure ECG data. The ECG measurement apparatus 10 may transmit ECG data to the first device 20.

In S140, the first device 20 may extract, from the ECG data, a first peak, which is the peak of P, the second peak, which is the peak of Q, the third peak, which is the peak of R, the fourth peak, which is the peak of S, and the fifth peak, which is the peak of T, and calculate values of the first to fifth peaks. The first to fifth peaks may be determined based on the features of each respectively peak that are collected by various algorithms. The values of the first to fifth peaks may be one of the voltage values or current values at the first to fifth peaks. The ECG data includes periodic waveforms, preferably including the peaks of P, Q, R, S, and T. The ECG data may be divided into signal segments in units of heartbeat cycles e.g., RR intervals. The first device 20 may detect the R peak having the highest value in the heartbeat and extract the first to fifth peaks using the peak detection module. Peak detection modules may be based on wavelet transform-based peak detection techniques, temporal characteristic-based detection techniques that detect valid peaks based on the relative position and interval between peaks, or other techniques. The first device 20 may output the time values of the first to fifth peaks and the magnitude values of voltage, current, etc. using the peak detection technique.

In the S150, the first device 20 may calculate the placement value based on the first peak value and the third peak value. The first peak value may be a value of P peak. The third peak value may be a value of R peak. The peak values may be a time value or a magnitude of peak in the PQRST cycle of the ECG data. The first device 20 may calculate the placement value using the placement determination module. The input of the placement determination module may be the first peak value or the third peak value. The placement determination module calculates the ratio of the first peak value and the third peak value, and if the ratio is within a preset threshold range, the placement state may be output as proper. At this time, the preset threshold range is set to the minimum and maximum values, and may be set above the minimum value and below the maximum value. The preset threshold range may vary depending on the ECG signal analysis technique or algorithm. The preset threshold range may be determined using an algorithm trained on previously ECG data. If the ratio of the first peak value and the third peak value is within the preset threshold range, the placement state is output as proper normal, attached, and if not, the placement state may be output as improper, abnormal, detached, loose, etc.

The placement determination module calculates the pattern of the ECG signal calculated based on the detected peak values, and may determine the placement state according to the pattern of the ECG signal. For example, a time interval between the first peak in the first cycle and the first peak in the second cycle can be calculated, and the time interval can be calculated in the subsequent cycles, and a degree of similarity between the time intervals may be used to determine whether the placement state is proper or improper. The placement determination module can determine the placement state as nonconforming if the time interval exceeds the reference multiple of the time interval in the preceding cycle, for example, by 1.3 times. In another embodiment, the placement determination module may analyze the temporal pattern of the detected peak values across multiple cardiac cycles. For example, the placement determination module may compute the time interval between the first peak (P wave) in successive cycles, and evaluate whether such intervals remain consistent across cycles. If the time interval in a given cycle deviates beyond a reference multiple (e.g., 1.3 times) of the preceding cycle's interval, the placement determination module may determine that the placement state is improper.

Based on peak values detected from the ECG data, the placement determination module may determine whether the placement state is proper or improper based on whether the Q, R, and S waves occur consecutively after the peak corresponding to the P wave. The placement determination module may determine whether the placement state is proper or improper based on at least one PQRST waveform feature. The PQRST waveform features may be created in the following way.

The placement determination module may determine the placement state based on a slope of the ST segment between the S-peak and the T-peak. The slope of the ST segment in the electrocardiogram signal measured from user is recognized as a pattern in which the slope is either descending, horizontal, or ascending, and the placement state may be determined as proper or improper based on whether the ST segment occurs according to the pattern in the electrocardiogram signal measured from user. The slope of the ST segment between the S-peak and the T-peak may be the PQRST waveform feature.

The placement determination module may calculate a placement value based on the first peak and the third peak. The placement determination module may determine the placement state based on the placement value. Additionally, the placement state may be determined based on one or more PQRST waveform features extracted from the ECG signal. The PQRST waveform features may include whether the time interval of the cardiac cycle is constant, whether the Q, R, and S waves occur continuously after the P wave, and at least one of the slopes of the ST segment. The PQRST waveform feature may be whether the time interval of the cardiac cycle is constant, whether the Q, R, and S waves occur continuously after the P wave, and at least one of the slopes of the ST segment. The PQRST waveform feature may be obtained by performing clustering on the electrocardiogram data.

Additionally, the placement determination module may determine the placement state by whether the ratio of the first peak value and the third peak value is within the preset threshold range. The placement determination module may determine the placement value or state upon receiving a user input without delay. The user input may be inputted in the ECG measurement apparatus 10, the first device 20, or the second device 30. The user input may be inputted in other external device.

The first device 20 may determine the placement state as either improper or proper based on the values of the first peak and the third peak. More specifically, the first device 20 may determine the placement value by whether the ratio value of the third peak value to the first peak value is above a preset threshold. If the ratio of the third peak value to the first peak value is greater than a preset threshold, a placement state is deemed to be proper. The first device 20 may display the placement value or the placement state.

In S160, the user may check the displayed data and if the placement state is proper, complete the placement of the electrocardiograph and process the electrocardiograph to measure the electrocardiogram signal. If the user checks the displayed data and the placement state is not proper, the user will change the placement position of the ECG measurement apparatus 10 and check the placement value again. The process of changing the position of the ECG measurement apparatus 10 is repeated until the placement state is proper. This allows the position of the ECG measurement apparatus 10 to be optimized. It prevents inaccurate measurements that occur when measuring ECG data due to differences in the user's physical characteristics.

The placement value may correspond to an index indicating whether an ECG measurement apparatus 10 is attached to a position that allows accurate detection of electrical signals generated by the heart. When the ECG measurement apparatus 10 is not properly attached to a position that allows accurate signal acquisition, one or more characteristic peaks corresponding to a cardiac cycle may be absent or distorted in the measured ECG signal.

In particular, the P wave typically exhibits a relatively low magnitude compared to the QRS complex and T wave, and may not be detected reliably when the placement state is improper. Therefore, whether the P wave is detected, and whether the first to fifth detected peak values respectively corresponding to the P, Q, R, S, and T waves are consistent with a physiologically valid cardiac cycle, may be used as a basis for determining whether the placement state is proper or improper.

The placement determination module may analyze the presence, sequence, amplitude, and timing of the first through fifth peaks to determine whether the ECG signal represents a valid PQRST pattern. If the pattern deviates from an expected physiological norm-such as by missing the P wave or exhibiting irregular intervals between peaks—the placement state may be determined to be improper. Conversely, if all five peaks are present and form a coherent pattern consistent with normal cardiac activity, the placement state may be determined as proper.

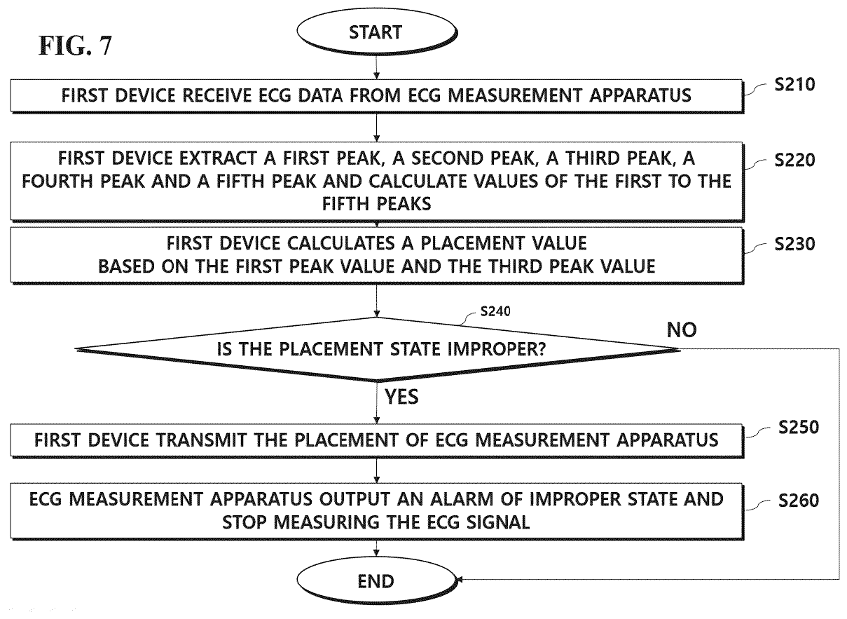

FIG. 7 is a flowchart illustrating a method for processing the placement state of ECG data during measurement according to one embodiment of the present disclosure. By outputting the placement state while the ECG measurement apparatus 10 is connected to the first device 20 and the ECG data is measured and transmitted to the first device 20, the ECG measurement apparatus 10 can prevent the electrocardiogram signal from being measured inaccurately. As the ECG measurement apparatus 10 is attached to the body for more than 14 days, the placement position may change due to user's ongoing movements. If the placement position of the ECG measuring apparatus 10 is changed in this way, the electrocardiogram signal may be measured in an improper state if it is moved to a position other than the position resulting in the known and consistent electrocardiogram signal.

Conventionally, the user has attached the ECG measurement apparatus 10 to the position indicated in a device manual and measured the ECG data for the specified measurement period. And, after the measurement was completed, the user has removed the ECG measurement apparatus 10 and has shipped it to a place for data analysis, and a data analyst obtained the electrocardiogram signal from the electrocardiograph 10. This process was used to measure and analyze the electrocardiogram signal. In such cases, it was possible to ensure whether the measurement of the electrocardiogram signal was proper in the analysis process by checking whether the ECG measurement apparatus 10 was placed to the proper position and measured.

If the ECG data was measured at the position, the user would have to go through the hassle of wearing the ECG measurement apparatus 10 for a set period of time again. This resulted in a decrease in the efficiency and user convenience of electrocardiogram measurement.

For the sake of convenience, the first device 20 is described in the following description, but the first device 20 may be replaced with the second device 30.

In S210, the first device 20 may receive ECG data from the ECG measurement apparatus 10.

In the S220, the first device 20 may extract the first peak, which is the peak of P, the second peak, which is the peak of Q, the third peak, which is the peak of R, the fourth peak, which is the peak of S, and the fifth peak, which is the peak of T, from each segmentation of the ECG data, and calculate the values of the first to fifth peaks. Each segmentation may have P, Q, R, S, T peaks. Each segmentation may represent a single time interval and may refer to a signal segment that includes one peak each of P, Q, R, S, and T. The values of the first to fifth peaks may be either the voltage values or the current values at the first to fifth peaks. The first device 20 may detect the R peak having the highest value in the heartbeat and extract the first to fifth peaks using the peak detection module. The peak detection module may be a wavelet transform-based peak detection technique, a temporal characteristic-based detection technique that detects valid peaks based on the relative position and interval between peaks, but may also use various other techniques. The first device 20 may output the placement value and/or the placement state. The first device 20 may apply a peak detection algorithm to an ECG signal and output one or more features associated with the first to fifth peaks, which correspond respectively to the P, Q, R, S, and T waves. The output features may include time values indicating the temporal positions of the respective peaks and magnitude values such as voltage, current, or other signal strength indicators corresponding to the detected peaks.

In the S230, the first device 20 may calculate a placement value based on the first peak value and the third peak value. The first peak value may be a value of P peak. The third peak value may be a value of R peak. The peak values may be time value or a magnitude of peak in the PQRST cycle of ECG data. The first device 20 may calculate the placement value using the placement determination module. The input of the placement determination module may be the first peak value and the third peak value. The placement determination module calculates the ratio of the first peak value to the third peak value, and if the ratio is within the preset threshold range, the placement state may be output as proper. The preset threshold range is set as the minimum and maximum values, and may be set above the minimum value and below the maximum value. If the ratio of the first peak value to the third peak value is within the critical range, the placement state is output as proper, normal, or attached. Otherwise, the placement state may be output as improper, abnormal, detached, or loose.

The first device 20 may determine the placement state as either proper or improper based on the values of the first peak and the third peak. More specifically, the first device 20 may determine the placement value by whether the ratio value of the third peak value to the first peak value is above a preset threshold. If the ratio value of the third peak to the first peak is greater than the preset threshold, the placement value is deemed to be proper. In S240 and S250, if the placement state is output as improper, the first device 10 may transmit the placement value to the ECG measurement apparatus 10. In the S260, the ECG measurement apparatus 10 may output an alarm of nonconformity and stop measuring the electrocardiogram signal.

FIG. 8 is a non-limiting example of a user interface in which ECG data and placement states are displayed according to one embodiment of the present disclosure. As shown in FIG. 8, the first device 20 or the second device 30 may output and display the placement state for the ECG data measured for each time interval for the received ECG data by way of example. The first device 20 or the second device 30 may output the placement state for the ECG data measured in real time using the peak detection module and the placement determination module. The first device 20 or the second device 30 may detect an event of an improper state for the ECG data.

The first device 20 or the second device 30 may determine the placement value for each ECG data of each heartbeat. The first device 20 or the second device 30 may be implemented to output an event in which the placement state changes from proper to improper or from improper to proper.

The first device 20 or the second device 30 may display a placement state as a proper state for the real-time measured ECG data in the first time period TP1. The first device 20 or the second device 30 may display a placement state as an improper state for the real-time measured ECG data in the second time period TP2. The first device 20 or the second device 30 may display a placement state as a proper state for the real-time measured ECG data in the third time period TP3. The first device 20 or the second device 30 may display a placement state as an improper state for the real-time measured ECG data in the fourth time period TP4. The first device 20 or the second device 30 may display a placement state as a proper state for the real-time measured ECG data in the fifth time period TP5.

The first device 20 may communicate with the ECG measurement apparatus 10 to receive the first ECG data and calculate the placement value for the first ECG data. The second device 30 may receive second ECG data and calculate the placement value for the second ECG data while the first device 20 is not communicating with the ECG measurement apparatus 10. The second device 30 may transmit the second ECG data and the placement value for the second ECG data to the first device 20 when communication between the first device 20 and the ECG measurement apparatus 10 is resumed. When communication with the ECG measurement apparatus 10 is resumed, the first device 20 may request and receive the second ECG data measured while it was not communicating with the second device 30. The first device 20 may receive the second ECG data and the placement value for the second ECG data from the second device 30, and display the second ECG data and the placement state by combining them with the first ECG data that it is storing.

According to one embodiment of the present disclosure, an external first device 20 or second device 30 may acquire ECG data measured by an ECG measurement apparatus 10 in real time and analyze the ECG data to detect the placement value of the ECG measurement apparatus 10 in real time. If the measurement is performed in an improper position, a warning or alarm may be generated to change the placement value of the ECG measurement apparatus 10.

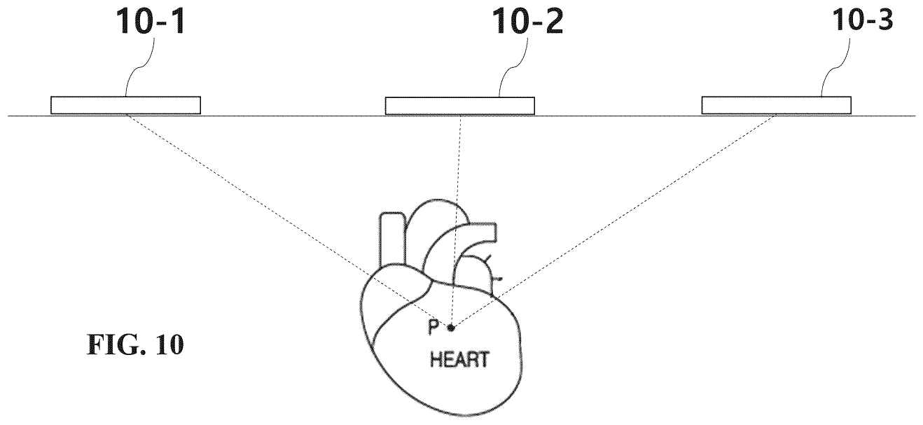

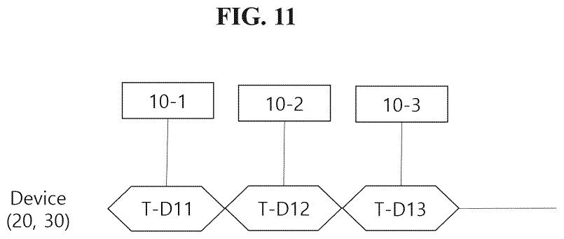

FIG. 9 is a schematic illustration of a process of measuring ECG data by changing a placement position of the ECG measuring device. FIG. 10 is a schematic illustration of the process by which an ECG measurement apparatus measures ECG data at each placement position. FIG. 11 is a schematic illustration of a method for receiving and processing ECG data measured at multiple positions.

First, the ECG measurement apparatus 10 may be positioned at the first position 10-1 near the heart and ECG data may be measured. After measuring the ECG data for a predetermined period of time, the ECG measurement apparatus 10 may be placed to a second position 10-2 and the ECG data may be measured. The ECG measurement apparatus 10 may be placed to a third position 10-3 and the ECG data may be measured again. The ECG measurement apparatus may be measured at multiple positions.

When the user applies the ECG measurement apparatus 10, the ECG measurement apparatus 10 may send a signal to the first device 20 or the second device 30 to notify placement states of the ECG data to start measuring, and place the ECG measurement apparatus 10 to different positions such as the first position, the second position, and the third position at a set time interval to measure the electrocardiogram signal.

The user places the ECG measurement apparatus 10-1 to the first position with a horizontal line and a specified angle θ according to the specified manual, and places the ECG measurement apparatus 10-2 to the second position with a specified distance d1 in the horizontal direction, The ECG measurement apparatus 10 may be positioned in the order of attaching the ECG measurement apparatus 10-3 to a third position having a predetermined distance d2 in the vertical direction. At this time, the predetermined angle θ may correspond to the angle between the boundary of the rib near the user's solar plexus and the horizontal line. The set distance di may be determined in response to the width of the user's chest. The set distance dl may be determined to be about 1/10 of the width of the chest. The distance d2 may be determined in response to the length between the user's shoulders and waist. The distance d2 may be determined to be about ⅕ to 1/7 of the length between the shoulders and waist. The set angle θ, distance d1, and distance d2 are not limited to these and may be determined in various ways.

According to one embodiment of the present disclosure, the user may identify a position where the ECG data is measured most accurately by providing information on the position with the best placement state by calculating the placement value while sequentially placing the ECG measurement apparatus 10 to a predetermined number of positions. It is described as being attached to three positions, but the ECG measurement apparatus 10 may be designed to be placed to two, five, or other positions.

The first device 20 or the second device 30 may receive ECG data for a fixed length of time at each placement position to calculate the placement value for each placement position and determine whether a placement state of the placement value is proper or improper.

As shown in FIG. 9, the user changes the placement position of the ECG measurement apparatus 10 to the first position 10-1, the second position 10-2, and the third position 10-3 so that ECG data is measured. The first position 10-1, second position 10-2, and third position 10-3 may be determined by a pre-set manual. The ECG data measured at the first position 10-1, second position 10-2, and third position 10-3 may have different shapes and values depending on a distance and angle from a heart of each user which is caused by the distribution of internal organs of each user, and each user's size and shapes.

The term “pre-set manual” as used herein refers to a predefined guide or instruction set configured to assist users in accurately positioning the ECG measurement apparatus at multiple locations on the body to obtain reliable and reproducible ECG data. The manual may be provided in various forms including, but not limited to, a printed guide, a graphical user interface displayed on a screen of the electronic device, or an interactive software application. The pre-set manual may define specific positions, such as the first position 10-1, second position 10-2, and third position 10-3, on the user's body based on anatomical landmarks. These positions may be determined through clinical research or empirical data to optimize signal acquisition from the heart while minimizing noise or interference caused by variations in body shape, internal organ distribution, and tissue conductivity. According to the pre-set manual, the first to third positions may be distinctively determined with reference to the location of the user's heart.

In one embodiment, the pre-set manual includes visual diagrams showing the relative placement of the ECG measurement apparatus in relation to the chest, sternum, or rib cage. The pre-set manual may further provide textual instructions or haptic feedback to guide the user during repositioning of the apparatus. Moreover, the manual may be customized based on user-specific parameters such as body mass index (BMI), age, gender, or medical history. In some implementations, the pre-set manual may be dynamically adjusted through an algorithm based on real-time feedback from previously measured ECG data, thereby improving measurement accuracy. The pre-set manual may include information on attachment positions of the device while providing a body illustration. The pre-set manual may include information on a first position, a second position, and a third position corresponding to body regions such as the chest, ribs, and abdomen. The second position may be represented as a position spaced a predetermined distance from the first position and having a predetermined angle relative to the first position. The third position may also be represented based on a distance and/or angular relationship. In addition to the first to third positions, the pre-set manual may provide additional exemplary attachment positions. The pre-set manual may be electronically provided to the first device 20 or the second device 30.

At each of the first to third positions, as shown in FIG. 10, the distance from the heart, direction, and the distribution and shape of organs from the heart to the skin are changed, and the ECG data measured are also changed. The ECG data measured at each of the first to third positions may have different values for the P, Q, R, S, and T peaks, and the shape and pattern of the signal may vary accordingly. Accordingly, the placement values of the ECG data measured at each of the first to third positions will also vary.

As shown in FIG. 11, in the first time period T-D11, ECG data is measured in the first position, in the second time period T-D12, ECG data is measured in the second position, and in the third time period T-D13, ECG data is measured in the third position, respectively. The first device 20 or the second device 30 may receive the ECG data.

The first device 20 or the second device 30 may receive the ECG data by setting the time intervals corresponding to the placement positions. The first device 20 or the second device 30 may receive ECG data while communicating with a single ECG measurement apparatus 10 and calculate the placement value for each time segment set for each placement position. In some embodiments, the first device 20 or the second device 30 may be configured to output data related to the first position 10-1, the second position 10-2, and the third position 10-3. The data related to the first position 10-1, the second position 10-2, and the third position 10-3 may be modified based on a user input received at the first device 20 or the second device 30. The first position, at which the ECG signal is measured during a first period, the second position, at which the ECG signal is measured during a second period, and the third position, at which the ECG signal is measured during a third period, may correspond to different placement positions of the ECG measurement apparatus 10. The first ECG data measured during the first period, the second ECG data measured during the second period, and the third ECG data measured during the third period may be output as time-series data. The time-series data may visually represent changes or differences in the ECG signals depending on the respective measurement positions and periods, thereby allowing for comparative analysis.

For example, output data indicating the first position 10-1, the second position 10-2, and the third position 10-3 within a body region of the user may be displayed by the first device 20 or the second device 30. In response to a user input, the positions at which the first position 10-1, the second position 10-2, and the third position 10-3 are displayed within the user's body region may be moved on the output data. Accordingly, updated output data reflecting the modified positions after the movement may be generated by the first device 20 or the second device 30.

According to one embodiment of FIG. 11, the first device 20 or the second device 30 may receive and display the ECG data in real-time. And a caregiver or user may decide the placement interactively by checking the ECG data displayed.

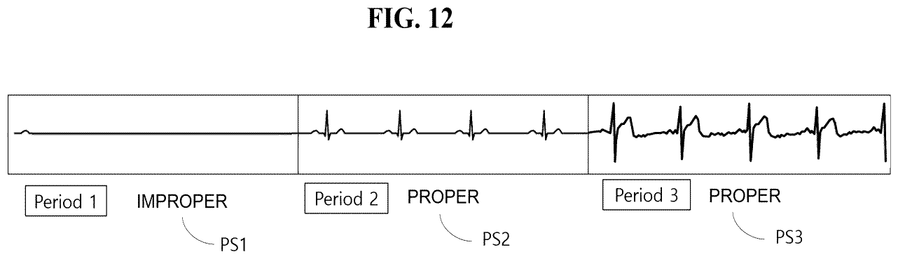

FIG. 12 depicts a non-limiting example of a user interface that displays ECG data and placement values for ECG data while being attached to multiple positions. As shown in FIG. 12, the first device 20 or the second device 30 has a first placement state PS1 for the ECG data placed to the first position in the first time period T-D11, a second placement state PS2 for the ECG data placed to the second position in the second time period T-D12, in the third time period T-D13, the third placement state PS3 for the ECG data placed to the third position may be output, and the best value among the first placement value PS1, the second placement state PS2, and the third placement state PS3 may be selected. As shown in FIG. 12, the best of the placement states may be the second placement state. The first device 20 or the second device 30 may display the first placement state PS1, the second placement state PS2, the third placement state PS3, and the selected information PS-2. The first device 20 or the second device 30 may display and store the electrocardiogram signals measured in each period, but it is not limited to that. Information on the best value among the first placement state PS1, the second placement state PS2, and the third placement state PS3 may include information on the number of the measured total number of ECG data, measured in the n-th place. Information such as “second placement state PS2” may be transmitted to the user's device. The user may measure the ECG data by applying the ECG measurement apparatus 10 to the position 10-2 with the best placement state among the first to third positions.

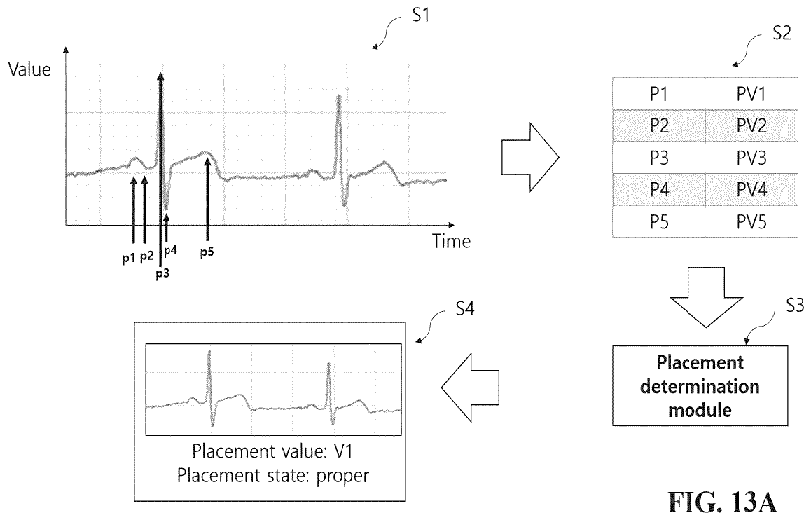

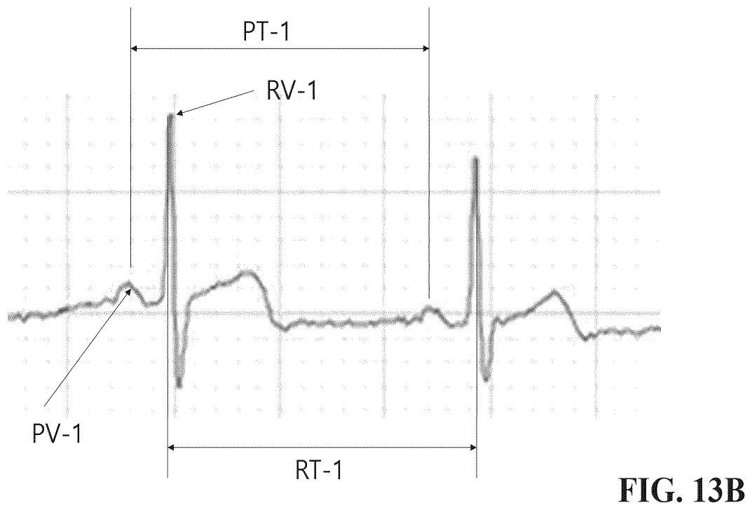

FIG. 13A is a schematic illustration of a process in which a placement state is calculated and displayed according to an embodiment of the present disclosure. FIG. 13B is a schematic illustration of a process of showing features detected by the placement determination module.

The first device 20 or the second device 30 may convert the measured electrocardiogram (ECG) signal into two-dimensional data consisting of time and signal amplitude. From the two-dimensional data, points corresponding to peaks (P1, P2, P3, P4, P5) may be extracted (S1). The peak points may be identified based on waveform characteristics or morphological features. For example, the R peak may be identified as the point having the highest amplitude, and the P peak may be identified as a point having a small amplitude that precedes the R peak. Accordingly, the peak points may be extracted based on their individual features.

The waveform characteristics or morphological features may have been learned from previously annotated ECG data (S2). Through machine learning, each waveform feature or morphological feature may be stored and managed.

The first device 20 or the second device 30 may obtain the peak values (PV1, PV2, PV3, PV4, PV5) corresponding to the peak points from the two-dimensional data. The first device 20 or the second device 30 may input the peak values into the placement determination module, which then calculates a placement value and a placement state for the corresponding time segment. (S3)

The placement determination module may calculate a placement value related to whether the ECG signal is measured from a proper attachment location, by using the amplitude value of the R peak, which has the largest amplitude, and the amplitude value of the P peak, which has the smallest amplitude (S3). The placement determination module may calculate the placement value using the R peak value and the P peak value among the five detected peaks, and determine the corresponding placement state (S4). The ratio between the amplitude value of the R peak and the amplitude value of the P peak reflects the difference in amplitude between the R peak and the P peak in the ECG signal. The ratio value between the R peak and the P peak may represent characteristics of each patient's cardiac motion and ECG waveform. In a proper state, the ECG signal may exhibit a consistent ratio between the amplitude value of the R peak and the amplitude value of the P peak. In a proper state, the ECG signal may also exhibit a consistent time interval between the time value of the R peak and the time value of the P peak. The time difference between the time value of the R peak and the time value of the P peak may be calculated as a placement value, and the placement state may be determined based on this value.

As illustrated in FIG. 13B, the placement determination module may also calculate the placement value using a different method. If it is determined that the characteristics of the patient's ECG signal are prominent in the amplitude values of the R and P peaks (e.g., RV-1 and PV-1), the placement determination module may calculate the placement value and determine the placement state using only the amplitude value of the R peak (RV-1) or only the amplitude value of the P peak (PV-1). The placement value may be calculated using the average amplitude value (threshold) of the P peaks measured from the ECG signal of the corresponding patient. Likewise, the placement value may be calculated using the average amplitude value (threshold) of the R peaks.

If it is determined that the distinguishing characteristics of the ECG signal appear in the timing values of the R and P peaks, the placement determination module may calculate the placement value using the time difference between a current R peak and a previous R peak (RT-1), or the time difference between a current P peak and a previous P peak (PT-1). The placement state may be determined by setting a threshold based on the time intervals between the R peaks measured from the ECG signal, or based on the time intervals between the P peaks. The placement determination module is not limited to the above methods and may calculate the placement value or placement state based on the peak values of the detected peaks. The placement determination module may collect features of the ECG signal specific to the corresponding patient and select one or more peak values, or relational expressions between a plurality of peak values, that show a high correlation with the placement state. The placement state may be determined by applying a rule or feature that has a significant correlation with the placement state. The first device 20 or the second device 30 may display the calculated placement value and placement state along with the ECG signal (S5). The ECG signal may be displayed together with the placement value and placement state.

What has been described above includes mere examples of various embodiments. It is, of course, not possible to describe every conceivable combination of components or methodologies for purposes of describing these examples, but one of ordinary skill in the art can recognize that many further combinations and permutations of the present embodiments are possible. Accordingly, the embodiments disclosed and/or claimed herein are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

Computing devices typically comprise a variety of media, which can comprise computer-readable storage media and/or communications media, which two terms are used herein differently from one another as follows. Computer-readable storage media can be any available storage media that can be accessed by the computer and comprises both volatile and nonvolatile media, removable and non-removable media. By way of example, and not limitation, computer-readable storage media can be implemented in connection with any method or technology for storage of information such as computer-readable instructions, program modules, structured data or unstructured data. Computer-readable storage media can comprise the widest variety of storage media including tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

In addition, a flow diagram may include a “start” and/or “continue” indication. The “start” and “continue” indications reflect that the steps presented can optionally be incorporated in or otherwise used in conjunction with other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

As may also be used herein, the term(s) “operably coupled to”, “coupled to”, and/or “coupling” includes direct coupling between items and/or indirect coupling between items via one or more intervening items. Such items and intervening items include, but are not limited to, junctions, communication paths, components, circuit elements, circuits, functional blocks, and/or devices. As an example of indirect coupling, a signal conveyed from a first item to a second item may be modified by one or more intervening items by modifying the form, nature or format of information in a signal, while one or more elements of the information in the signal are nevertheless conveyed in a manner than can be recognized by the second item. In a further example of indirect coupling, an action in a first item can cause a reaction on the second item, as a result of actions and/or reactions in one or more intervening items.

Although specific embodiments have been illustrated and described herein, it should be appreciated that any arrangement which achieves the same or similar purpose may be substituted for the embodiments described or shown by the subject disclosure. The subject disclosure is intended to cover any and all adaptations or variations of various embodiments. Combinations of the above embodiments, and other embodiments not specifically described herein, can be used in the subject disclosure. For instance, one or more features from one or more embodiments can be combined with one or more features of one or more other embodiments. In one or more embodiments, features that are positively recited can also be negatively recited and excluded from the embodiment with or without replacement by another structural and/or functional feature. The steps or functions described with respect to the embodiments of the subject disclosure can be performed in any order. The steps or functions described with respect to the embodiments of the subject disclosure can be performed alone or in combination with other steps or functions of the subject disclosure, as well as from other embodiments or from other steps that have not been described in the subject disclosure. Further, more than or less than all of the features described with respect to an embodiment can also be utilized.

What is claimed is:

1. An electrocardiogram measurement system comprising:
an electrocardiogram measurement apparatus configured to measure an electrocardiogram data via means of one or more electrodes, amplify the electrocardiogram data, digitize the electrocardiogram data, and transmit the amplified and digitized electrocardiogram data through a first communication unit; and
a first device comprising a first processor, a second communication unit, a third communication unit and a first memory, the first device configured to:
receive a first electrocardiogram data and a first time value from the electrocardiogram measurement apparatus via the second communication unit during a first period,
receive a second electrocardiogram data and a second time value from the electrocardiogram measurement apparatus via the second communication unit during a second period,
receive a third electrocardiogram data and a third time value from the electrocardiogram measurement apparatus via the second communication unit during a third period,
extract, respectively, values of P peaks and values of R peaks from each of the first to the third electrocardiogram data using a peak detection module,
determine a first placement state using a first value of the P peak and a first value of the R peak in a PQRST cycle of the first electrocardiogram data using a placement determination module,
determine a second placement state using a second value of P Peak and a second value of R peak in a PQRST cycle of the second electrocardiogram data using the placement determination module,
determine a third placement state using a third value of P Peak and a third value of R peak in a PQRST cycle of the third electrocardiogram data using the placement determination module, and
display the first, the second, and the third placement states through an output unit.

2. The electrocardiogram measurement system of claim 1, wherein the first device is further configured to:
receive a fourth electrocardiogram data after the third period has elapsed and after a predetermined time,
determine a fourth placement state for one or more signal segments included in the fourth electrocardiogram data, and
generate and output an alert upon detecting an event where the fourth placement state indicates an improper attachment state of the electrocardiogram measurement apparatus.

3. The electrocardiogram measurement system of claim 2, wherein the first device is further configured to:
transmit a request of the alert to the electrocardiogram measurement apparatus.

4. The electrocardiogram measurement system of claim 1, further comprising a second device,
wherein the second device comprising a second processor, a fourth communication unit, a fifth communication unit and a second memory, and is configured to:
communicate with the first device wirelessly upon request from the first device;
receive a fifth electrocardiogram data and measured time information from the electrocardiogram measurement apparatus via the fourth communication unit in response to a command for making a connection with the electrocardiogram measurement apparatus from the first device via the fifth communication unit;
store the fifth electrocardiogram data measured by the electrocardiogram measurement apparatus over a fifth period in the second memory;
determine a fifth placement state for one or more signal segments included in the fifth electrocardiogram data; and
generate and output an alert upon detecting an event where the fifth placement state indicates an improper placement state including a detachment of the electrocardiogram measurement apparatus; and
wherein:
the electrocardiogram measurement apparatus transmits the fifth electrocardiogram data to the second device via the first communication unit while the electrocardiogram measurement apparatus is not communicating with the first device; and
a fifth time period refers to a duration during which the second device is communicating with the electrocardiogram measurement apparatus and while the first device is not communicating with the electrocardiogram measurement apparatus.

5. The electrocardiogram measurement system of claim 4, wherein the second device transmits a request of the alert to the electrocardiogram measurement apparatus.

6. The electrocardiogram measurement system of claim 4, wherein the first device is further configured to:
receive the fifth electrocardiogram data and the fifth placement state for one or more signal segments included in the fifth electrocardiogram data from the second device; and
display the fifth placement state of the fifth electrocardiogram data through a display device.

7. The electrocardiogram measurement system of claim 1, wherein:
the first electrocardiogram data is measured when the electrocardiogram measuring apparatus attached to a first placement position,
the second electrocardiogram data is measured when the electrocardiogram measuring apparatus attached to a second placement position,
the third electrocardiogram data is measured when the electrocardiogram measuring apparatus attached to a third placement position, and
the first to third placement positions are determined relative to a user's heart according to instructions specified in a pre-set manual.

8. The electrocardiogram measurement system of claim 1, wherein the peak detection module is configured to extract values of P peaks and values of R peaks from electrocardiogram data using a wavelet transform function.

9. The electrocardiogram measurement system of claim 1, wherein the placement determination module determines the first placement state, the second placement state, the third placement state, by applying a predetermined algorithm that is based on the first, the second and the third values of the P peaks and the R peaks in the first to third electrocardiogram data, wherein the predetermined algorithm is trained on previously measured data.

10. The electrocardiogram measurement system of claim 9, wherein the predetermined algorithm is further based on one or more PQRST waveform features, wherein the one or more PQRST waveform features are obtained by performing clustering on the electrocardiogram data.

11. The electrocardiogram measurement system of claim 1, wherein the placement determination module is configured to determine a fourth placement state upon receiving a user input without delay.
